(19) United States
(12) Patent Application Publication
(10) Pub. No.: US 2026/0076891 A1
(43) Pub. Date: Mar. 19, 2026

(54) POLYORGANOSILOXANE EMULSIONS WITH LARGE PARTICLE SIZE AND PROCESS FOR THEIR PREPARATION

(71) Applicant: Wacker Chemie AG, Munich (DE)

(72) Inventors: Holger Rautschek, Munich (DE); Gerhard Beer, Munich (DE); Steffen Manemann, Munich (DE)

(73) Assignee: Wacker Chemie AG, Munich (DE)

(21) Appl. No.: 19/106,227

(22) PCT Filed: Mar. 3, 2023
(86) PCT No.: PCT/EP2023/055442

§ 371 Date: Feb. 25, 2025

(51) Int. Cl. A61K 8/891; A61K 8/06; A61Q 5/02

(52) U.S. Cl. CPC A61K 8/891; A61K 8/062; A61Q 5/02

(57) ABSTRACT

A process for preparing storage-stable, aqueous polyorganosiloxane emulsions includes preparing an emulsifier-water mixture that includes at least one alkyl polyglycoside (B) and water (C) and optionally further emulsifiers (D), optionally thickeners (E) and optionally further additives (F). The process also includes preparing a preliminary emulsion by addition of at least one polyorganosiloxane (A) to the emulsifier-water mixture. The preliminary emulsion is prepared under the action of low shear forces using stirring devices with a maximum peripheral speeds of 4.5 m/s. The process yields polyorganosiloxane emulsions having a particle size D(50) of 1 μm to 50 μm, based on the D(50) value of the volume distribution measured in accordance with the principle of Fraunhofer diffraction (according to ISO 13320).

The invention relates to storage-stable, aqueous polyorganosiloxane emulsions of relatively high-viscosity polyorganosiloxanes that contain alkyl polyglycosides as emulsifiers and have a large particle size, and to processes for preparing these emulsions.

Polyorganosiloxanes have a wide variety of uses. The terms “polyorganosiloxane” and “silicone” are used synonymously in the present invention. In order to facilitate application and metering particularly in the case of viscous products, it is desirable for many applications that the organosilicon compounds are in diluted form. It is possible to use organic solvents such as benzene or chlorohydrocarbons for this purpose, however this is disadvantageous from an ecological and occupational health point of view. Therefore, use is mostly effected in the form of aqueous emulsions or dispersions, usually as oil-in-water emulsions, which can be diluted with water. The oil phase here is understood to mean the water-immiscible organosilicon compounds, optionally dissolved in organic solvents.

Emulsions containing polyorganosiloxanes and other organosilicon compounds and processes for the preparation thereof have long been known from the prior art and are described, for example, in the textbook W. Noll, Chemistry and Technology of Silicones, 1968, p. 428-431, Verlag Chemie Weinheim.

Emulsifiers used for polyorganosiloxane emulsions are typically ethoxylated, synthetic alcohols, ethoxylated fatty alcohols, ethoxylated triglycerides, ethoxylated fatty acids and mixtures thereof (for example in EP 200916 B1, EP0463431 A2 and DE 19620405 B4).

Emulsifiers based on polyethylene glycol ethers and esters are undesirable in particular in cosmetic applications due to potential skin irritations (for example in US2007178144 AA (corresponding to EP1813251 A2)). In addition, there is an increased demand for products that contain emulsifiers based on renewable raw materials.

Alkyl polyglycosides are emulsifiers that are prepared from renewable raw materials and do not contain any polyethylene glycol groups.

It is known that silicone oils, for example polydimethylsiloxanes, can be emulsified with alkyl polyglycosides (for example in U.S. Pat. No. 5,133,897 A (corresponding to EP0418479 A1)). These emulsions can be prepared with low shear forces. Use is made of 2 to 40 parts of emulsifier, preferably 5 to 30 parts of emulsifier, based on the oil phase. In the examples, silicone oils with up to 2000 mPa·s are emulsified, with use being made of at least 11 parts of emulsifier per 100 parts of polyorganosiloxane in order to prepare stable emulsions.

If silicone defoamers, i.e. for example mixtures of silicone oil and finely divided silica, are to be emulsified, relatively high surfactant contents are required and the emulsion additionally has to be stabilized with thickeners or alcohols (see EP0769548 A1 and EP0774503 A1). EP0769548 A1 uses at least 5 parts, preferably at least 28 parts, of emulsifier based on the silicone oil. EP0774503 A1 likewise uses at least 5 parts, preferably at least 10 parts, of emulsifier based on the polyorganosiloxane. The examples of these patent specifications respectively use 25 parts and 50 parts of emulsifier per 100 parts of silicone oil/SiO₂.

However, high proportions of emulsifiers are economically disadvantageous in particular for cost reasons.

DE102014212725 A1 describes a process for the preparation of particularly finely divided emulsions based on high-viscosity silicone oils and alkyl polyglycosides. Particle sizes of less than 300 nm are achieved through the use of high shear forces. These emulsions are thus stable even at relatively low emulsifier concentrations of only about 12 parts of emulsifier per 100 parts of silicone oil. However, this process has the disadvantage that it requires appropriate units that can generate high shear forces in high-viscosity phases, which additionally means a high energy consumption and is therefore ecologically and economically disadvantageous.

For specific applications in cosmetics, preference is given to large particle sizes (as in WO 03/092639 A1). However, the tendency toward separation increases with the particle size in accordance with Stokes' law (cf. in this respect H. Schubert et al., Chem. Ing. Techn. 61, 1989, p. 701-711).

The object was therefore to provide emulsions based on high-viscosity silicone oils and a process for preparing these polyorganosiloxane emulsions, which contain emulsifiers based on renewable raw materials and have excellent stability even with very low emulsifier proportions and large particle sizes.

This object is achieved by the present invention.

The invention provides a process for preparing storage-stable, aqueous polyorganosiloxane emulsions comprising
(A) at least one polyorganosiloxane of the general formula

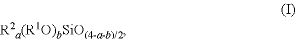

where
R¹ may be identical or different and is a hydrogen atom or a monovalent, substituted or unsubstituted hydrocarbon radical,
R² may be identical or different and is a monovalent, substituted or unsubstituted hydrocarbon radical having 1 to 30 carbon atoms or a hydrogen atom,
a is 0, 1, 2 or 3,
b is 0, 1, 2 or 3,
with the proviso that the sum total of a+b is ≤3,
(B) at least one alkyl polyglycoside of the general formula

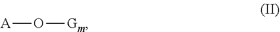

where
A is a linear or branched, saturated or unsaturated alkyl radical having 8 to 24 carbon atoms,
G is a glycoside radical and
m is the degree of glycosidation and is an integer of on average 1 to 5,
(C) water,
optionally
(D) further emulsifiers that are different from the alkyl polyglycosides (B), optionally
(E) thickeners
and optionally
(F) further additives that are usually present in polyorganosiloxane emulsions, characterized in that
(i) a total of 1.5 to 4.5 parts of alkyl polyglycoside (B) are present per 100 parts of polyorganosiloxane (A),
(ii) a total of 0 to 3 parts of further emulsifiers (D) are present per 100 parts of polyorganosiloxane (A),
(iii) the sum total of the parts of alkyl polyglycoside (B) and further emulsifiers (D) is ≤4.5 per 100 parts of polyorganosiloxane (A)
and
(iv) the process comprises the following steps
(1) preparation of an emulsifier-water mixture comprising at least one alkyl polyglycoside (B) and water (C) and optionally further emulsifiers (D), optionally thickeners (E) and optionally further additives (F),
(2) preparation of a preliminary emulsion by addition of at least one polyorganosiloxane (A) to the emulsifier-water mixture, with the proviso that the preliminary emulsion is prepared under the action of low shear forces using stirring devices with maximum peripheral speeds of 4.5 m/s, optionally
(3) dilution of the preliminary emulsion by addition of further water (C) and optionally
(4) addition of further additives (F),
where the process yields polyorganosiloxane emulsions having a particle size D(50) of 1 μm to 50 μm.

The invention further provides storage-stable, aqueous polyorganosiloxane emulsions comprising
(A) at least one polyorganosiloxane of the general formula

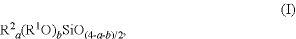

where
R¹ may be identical or different and is a hydrogen atom or a monovalent, substituted or unsubstituted hydrocarbon radical,
R² may be identical or different and is a monovalent, substituted or unsubstituted hydrocarbon radical having 1 to 30 carbon atoms or a hydrogen atom,
a is 0, 1, 2 or 3,
b is 0, 1, 2 or 3,
with the proviso that the sum total of a+b is ≤3,
(B) at least one alkyl polyglycoside of the general formula

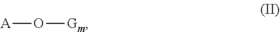

where
A is a linear or branched, saturated or unsaturated alkyl radical having 8 to 24 carbon atoms,
G is a glycoside radical and
m is the degree of glycosidation and is an integer of on average 1 to 5,
(C) water,
optionally
(D) further emulsifiers that are different from the alkyl polyglycosides (B),
optionally
(E) thickeners
and optionally
(F) further additives that are usually present in polyorganosiloxane emulsions, characterized in that
(i) a total of 1.5 to 4.5 parts of alkyl polyglycoside (B) are present per 100 parts of polyorganosiloxane (A),
(ii) a total of 0 to 3 parts of further emulsifiers (D) are present per 100 parts of polyorganosiloxane (A),
(iii) the sum total of the parts of alkyl polyglycoside (B) and further emulsifiers (D) is ≤4.5 per 100 parts of polyorganosiloxane (A)
and
(iv) the emulsions are produced by a process comprising the steps
(1) preparation of an emulsifier-water mixture comprising at least one alkyl polyglycoside (B) and water (C) and optionally further emulsifiers (D), optionally thickeners (E) and optionally further additives (F),
(2) preparation of a preliminary emulsion by addition of at least one polyorganosiloxane (A) to the emulsifier-water mixture, with the proviso that the preliminary emulsion is prepared under the action of low shear forces using stirring devices with maximum peripheral speeds of 4.5 m/s,
optionally
(3) dilution of the preliminary emulsion by addition of further water (C)
and optionally
(4) addition of further additives (F),
with the proviso that the polyorganosiloxane emulsions have a particle size D(50) of greater than 1 μm to at most 50 μm.

In order not to create an excessive number of pages in the description of the present invention, only the preferred embodiments of the individual features are specified hereinbelow.

However, the expert reader should explicitly understand this manner of disclosure such that every combination of different levels of preference is thus also explicitly disclosed and explicitly desired.

Preferably, polydimethylsiloxanes having a viscosity of 10 000 mPa·s to 3 000 000 mPa·s (determined at 25° C. in accordance with DIN 53019) are used as polyorganosiloxanes (A).

Preferably, alkyl polyglycosides (B) having a saturated alkyl radical having an average of 8 to 14 carbon atoms and an average degree of glycosidation m of 1.1 to 3.0 are used.

In a preferred embodiment, further emulsifiers (D) are not present.

Preferably, 0.1 to 1 part(s) of xanthan gum per 100 parts of polyorganosiloxane (A) are used as thickener (E).

Preferably, the instability index of the polyorganosiloxane emulsions, after centrifugation at 2300×g for 8 h, is <0.1.

In a preferred embodiment, the polyorganosiloxane emulsions have a particle size D(50) of 3 μm to 20 μm.

Preferably, the polyorganosiloxane emulsions have a content of octamethylcyclotetrasiloxane (D₄) of less than 0.1% by weight based on the total weight of the polyorganosiloxane emulsion.

The polyorganosiloxane emulsions according to the invention and the process according to the invention for the preparation thereof are described in more detail below:

Components (A) to (F) used in the process according to the invention may each be one type of such a component or else a mixture of at least two types of a respective component.

### Polyorganosiloxanes (A)

The polyorganosiloxanes (A) of the general formula (I) that are used in the emulsions according to the invention are preferably liquid at 25° C. and preferably have viscosities, measured at 25° C. in accordance with DIN ISO 53019, of 10 to 50 000 000 mPa·s and particularly preferably of 10 000 to 3 000 000 mPa·s.

The polyorganosiloxanes (A) are preferably those containing 5 to 10 000 units of formula (I), particularly preferably those consisting of units of formula (I) having an average value of a of 1.9 to 2.3 and an average value of b of 0 to 0.2. R¹ may be identical or different and is a hydrogen atom or a monovalent, substituted or unsubstituted hydrocarbon radical, preferably a hydrogen atom or a monovalent, substituted or unsubstituted hydrocarbon radical having 1 to 4 carbon atoms and particularly preferably a hydrogen atom.

R² may be identical or different and is a monovalent, substituted or unsubstituted hydrocarbon radical having 1 to 30 carbon atoms or a hydrogen atom.

Preferably, in the units of formula (I), a maximum of one radical R² has the definition of a hydrogen atom.

Radical R² is preferably a hydrogen atom or a monovalent, substituted or unsubstituted hydrocarbon radical having 1 to 18 carbon atoms, particularly preferably a hydrogen atom or the methyl or phenyl radical.

In a preferred embodiment, in particular 80 mol % or more of the radicals R² in the polyorganosiloxane (A) have the definition of methyl radicals and 20 mol % or less have the definition of aminopropyl or aminoethylaminopropyl radicals.

Non-exhaustive examples of hydrocarbon radicals R¹ or R² are alkyl radicals such as the methyl, ethyl, n-propyl, isopropyl, 1-n-butyl, 2-n-butyl, isobutyl, tert-butyl, n-pentyl, isopentyl, neopentyl, tert-pentyl radical, hexyl radicals such as the n-hexyl radical, heptyl radical such as the n-heptyl radical, octyl radicals such as the n-octyl radical and isooctyl radicals such as the 2,2,4-trimethylpentyl radical, nonyl radicals such as the n-nonyl radical, decyl radicals such as the n-decyl radical, dodecyl radicals such as the n-dodecyl radical, octadecyl radicals such as the n-octadecyl radical, cycloalkyl radicals such as the cyclopentyl, cyclohexyl and cycloheptyl radical and methylcyclohexyl radical, alkenyl radicals such as the vinyl, 1-propenyl and 2-propenyl radical, aryl radicals such as the phenyl, naphthyl, anthryl and phenanthryl radical, alkaryl radicals such as o-, m-, p-tolyl radicals, xylyl radicals and ethylphenyl radicals and aralkyl radicals such as the benzyl radical and the α- and β-phenylethyl radical.

Non-exhaustive examples of substituted radicals R¹ or R² are hydrocarbon radicals substituted by halogen, cyano, glycidoxy, polyalkylene glycol or amino groups, such as hydrocarbon radicals bearing trifluoropropyl, cyanoethyl, glycidoxypropyl, polyalkylene glycol propyl and amino groups.

Non-exhaustive examples of amino group-substituted radicals R² are:

H₂N(CH₂)₂NH(CH₂)₃—

H₂N(CH₂)₂—

H₃CNH(CH₂)₃—

H₂N(CH₂)₄—

H₂N(CH₂)₅—

H(NHCH₂CH₂)₃—

C₄H₉NH(CH₂)₂NH(CH₂)₂— and

cyclo-C₆H₁₁NH(CH₂)₃—,
where
H₂N(CH₂)₃— and
H₂N(CH₂)₂NH(CH₂)₃— are particularly preferred.

In formula (I), the sum total of a+b is ≤3 and has a value of preferably on average 1.5 to 2.4, particularly preferably on average 1.9 to 2.3 and very particularly preferably on average 1.95 to 2.05.

Non-exhaustive examples of the polyorganosiloxanes (A) used in the emulsions according to the invention are aminopropyldimethylsiloxy-, aminoethylaminopropyldimethylsiloxy-, vinyldimethylsiloxy-, trimethylsiloxy-, dimethylalkoxysiloxy group- or dimethylhydroxy group-terminated polydiorganosiloxanes, in particular polydimethylsiloxanes, containing pendant aminopropyl, aminoethylaminopropyl or carboxyalkyl groups.

Preferred polyorganosiloxanes (A) are those of the formula

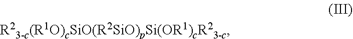

where
R¹ and R² have the definition given for them above,
c is 0, 1 or 2, preferably 0 or 1,
and
p is 1 to 100 000, preferably 5 to 10 000.

Non-exhaustive examples of preferred polyorganosiloxanes (A) used in the process according to the invention are:

(CH₃)₃Si—O—[Si(CH₃)₂—O—]ₙ—Si(CH₃)₃

HO(CH₃)₂Si—O—[Si(CH₃)₂—O—]ₙ—Si(CH₃)₂OH

(CH₃)₃Si—O—[Si(CH₃)₂—O—]ₙ—[Si(CH₃)R—O—]ₘ—Si(CH₃)₃

HO(CH₃)₂Si—O—[Si(CH₃)₂—O—]ₙ—[Si(CH₃)R—O—]ₘ—Si(CH₃)₂OH

CH₃O(CH₃)₂Si—O—[Si(CH₃)₂—O—]ₙ—[Si(CH₃)R—O—]ₘ—Si(CH₃)₂OCH₃

C₂H₅O(CH₃)₂Si—O—[Si(CH₃)₂—O—]ₙ—[Si(CH₃)R—O—]ₘ—Si(CH₃)₂OC₂H₅,
where
n is 50 to 5000,
m is 1 to 100,
and
R is for example a radical of the formula —C₃H₆NH₂, —C₃H₆NH—C₂H₄NH₂ or —C₁₀H₂₀COOH.

If a reactive polyorganosiloxane, for example with vinyl or OH end groups, is preferably used as polyorganosiloxane (A), then for example chain extension, polycondensation or, in the presence of suitable reactants, crosslinking may be effected in the emulsion. Reactions of this kind are known to those skilled in the art and result in the emulsions prepared according to the invention containing polyorganosiloxanes with viscosities of greater than 1 000 000 mPa·s (determined at 25° C. in accordance with DIN 53019) or crosslinked polyorganosiloxane elastomers.

The polyorganosiloxanes (A) used in the emulsions according to the invention are preferably prepared by the relevant processes in the literature.

### Alkyl Polyglycosides (B)

In a preferred embodiment, the alkyl polyglycosides (B) are compounds of the general formula (II),
where
A has the definition given above,
G is a hexose or pentose unit or mixtures thereof
and
m has the definition given above.

Compounds of this kind are described for example in U.S. Pat. No. 5,133,897 A (corresponding to EP0418479 A1).

The alkyl polyglycosides (B) may be characterized by 1H NMR spectroscopy, for example on a Bruker Avance 500 MHz NMR spectrometer, where the average chain length of the alkyl radical A or the average degree of glycosidation m can be determined by integration of the areas under the 1H NMR signals, as is known to those skilled in the art and as described for example in the textbook H. Fribolin, Ein-und zweidimensionale NMR-Spektroskopie [One- and Two-Dimensional NMR Spectroscopy], 5th Edition, 2013, p. 35, Wiley-VHC-Verlag and as processed for example according to the operating instructions of the Bruker Topspin 3.2 NMR data analysis software.

Preferably, the alkyl polyglycosides (B) are those having a saturated alkyl radical having an average of 8-14 carbon atoms and an average degree of glycosidation of 1.1 to 3.0.

Particular preference is given to alkyl polyglycosides (B) based on fatty alcohols having 8-10 carbon atoms and an average degree of glycosidation of 1.4 to 2.0.

Non-exhaustive examples of alkyl polyglycosides (B) are D-glucopyranose, oligomers, decyl octyl glycosides available under the trade name Disponil® APG 215 from BASF SE or under the trade name TRITON® CG 110 from Stockmeier GmbH, D-glucopyranose, oligomeric, C10-16-alkyl glycosides available under the trade name Glucopon® 650 EC and Glucopon® GD 70 from BASF SE.

### Water (C)

The water (C) used may be all types of waters that have also been used to date for the preparation of polyorganosiloxane emulsions.

The water (C) used is preferably partially or fully demineralized water, distilled or (repeatedly) redistilled water, water for medical or pharmaceutical purposes, such as purified water (Aqua purificata in accordance with Pharm. Eur.).

The water (C) used according to the invention preferably has a conductivity of less than 50 μS/cm, particularly preferably less than 10 μS/cm, in particular less than 1.3 μS/cm, in each case at 25° C. and 1010 hPa.

Water (C) is used in amounts of preferably 10 to 1000 parts by weight, particularly preferably 40 to 500 parts by weight, based on 100 parts by weight of polyorganosiloxane (A).

### Further Emulsifiers (D)

The polyorganosiloxane emulsions according to the invention may optionally comprise further emulsifiers (D). Use may be made in this case of all nonionogenic, anionic or cationic emulsifiers known to those skilled in the art. Preference is given here to emulsifiers based on renewable raw materials.

The further emulsifiers (D) are preferably commercially available emulsifiers. Non-exhaustive examples of substance classes of further emulsifiers (D) are sorbitan esters, glycerol esters, polyglycerol esters, alkyl sulfates and alkyl phosphates.

Preferably, the optionally used further emulsifiers (D) according to the invention may be used in pure form or as a solution of one or more emulsifiers in water or organic solvents.

### Thickeners (E)

As component (E), the emulsions according to the invention may optionally comprise thickeners; these are preferably cellulose ethers and polysaccharides, such as xanthan gum, particularly preferably xanthan gum.

If thickeners (E) are used, the amounts are preferably 0.01 to 2 parts based on 100 parts of polyorganosiloxane (A).

### Further Additives (F)

In addition to components (A) to (E), use may optionally be made of further additives (F) that are usually added to silicone emulsions.

Non-exhaustive examples of further additives (F) are further siloxanes that are different from the polyorganosiloxanes (A) such as polyethersiloxanes or silanes, in particular alkoxysilanes, fillers, additives such as preservatives, disinfectants, wetting agents, corrosion inhibitors, dyes and fragrances, and mixtures thereof.

If further siloxanes that are different from the polyorganosiloxanes (A) are used, they are preferably used in amounts of 0.1 to 1 parts based on 100 parts of polyorganosiloxane (A).

If silanes are used, they are preferably used in amounts of 0.1 to 50 parts based on 100 parts of polyorganosiloxane (A).

Non-exhaustive examples of additives that may be used according to the invention are preservatives, dyes or fragrances known those skilled in the art, in particular preservatives such as methylisothiazolinone, chloromethylisothiazolinone, benzylisothiazolinone, phenoxyethanol, methylparaben, ethylparaben, propylparaben, butylparaben, isobutylparaben, alkali metal benzoates, benzoic acid, alkali metal sorbates, sorbic acid, iodopropynyl butylcarbamate, benzyl alcohol and 2-bromo-2-nitropropane-1,3-diol.

If additives are used, the amounts are preferably 0.0005 to 2 parts based on 100 parts of polyorganosiloxane (A). Additives (F), in particular preservatives, are preferably used in the process according to the invention.

The emulsions according to the invention have particle sizes (median of the volume distribution) of 1 μm to 50 μm, in particular particle sizes of 3 μm to 20 μm, these figures being based on the D(50) value of the volume distribution measured in accordance with the principle of Fraunhofer diffraction (according to ISO 13320). A parameter assumed for the measurement is a spherical model with a refractive index of the continuous phase of 1.33, a refractive index of the disperse phase of 1.39 and an absorption of 0.01.

The emulsions prepared according to the invention preferably have a content of nonvolatile fractions, measured in accordance with DIN EN ISO 3251, of preferably 1% to 80% by weight, particularly preferably of 50% to 70% by weight.

The pH of the emulsions prepared according to the invention is preferably 3 to 10, particularly preferably 3 to 8.

Storage-stable emulsions are those emulsions in which no visible separation into two phases occurs after storage for 30 days at 50° C., preferably in a drying cabinet.

Preferably, the polyorganosiloxane emulsions according to the invention may be prepared batchwise or continuously, for example in accordance with US2018193808 (equivalent to EP3349888 B1).

The peripheral speed v (speed of a point on the edge of the rotor or disk) (in meters per second) is calculated by the following formula

$$v = U \cdot Π \cdot {d}_{R} , \qquad ( IV )$$

where
U is the rotational speed (in revolutions per second)
and
d_{R} is the diameter of the dispersing tool (in meters).

The greater the peripheral speed v, the stronger the shear forces acting on the product to be dispersed.

According to the invention, peripheral speeds v should be selected that are at most 4.5 m/s, preferably at most 3 m/s, particularly preferably at most 2 m/s and very particularly preferably at most 1 m/s.

Preferably, the process according to the invention is carried out at a pressure of the surrounding atmosphere, i.e. at about 1010 hPa.

The process according to the invention is preferably carried out at temperatures of 0° C. to 80° C., preferably of 2° C. to 50° C., and particularly preferably of 5° C. to 30° C.

Preferably, the process according to the invention is carried out at room temperature, i.e. about 25° C., or a temperature which is established on combination of the components at room temperature without additional heating or cooling.

The present invention further provides for the use of the polyorganosiloxane emulsions prepared by the process according to the invention in cosmetic compositions such as body or hair care products.

The emulsions prepared according to the invention can in principle be used for all other purposes for which emulsions having polyorganosiloxanes have also been used to date, for example as release agents, lubricants, hydrophobizing agents and for textile impregnation, in the processing of rubber and plastics or in metal processing and as hydrophobizing agents for glass and mineral building materials.

The process according to the invention has the advantage that, despite very low proportions of emulsifier and the action of low shear forces, it is possible to obtain very storage-stable and high-performance emulsions having average particle sizes of 1 μm to 50 μm, in particular of 3 μm to 20 μm (D(50) value of the volume distribution). This makes the process according to the invention energetically favorable and therefore economically and ecologically advantageous.

The large particle sizes, obtainable via the process according to the invention, of the polyorganosiloxane emulsions according to the invention further offer particular performance advantages in cosmetic hair care products.

The use of alkyl polyglycosides (B) as emulsifiers based on renewable raw materials is resource-friendly. Furthermore, the use of polysaccharide-based emulsifiers in cosmetic applications makes it possible to minimize potential skin irritations and intolerances that are caused by conventional emulsifiers based on polyethylene glycol ethers and esters.

In addition, the polyorganosiloxane emulsions according to the invention have excellent performance properties, such as a good conditioning effect in hair care products, i.e. significant reduction in the wet and dry combing force, and a good effect as release agent and lubricant, and a good wetting capacity on various substrates.

### EXAMPLES

The subject matter of the present invention will be elucidated by the examples which follow, but without limiting the invention to the contents disclosed therein. In the examples which follow, all figures for parts and percentages, unless stated otherwise, are based on weight. Unless stated otherwise, the examples which follow are conducted at a pressure of the surrounding atmosphere, i.e. at about 1010 hPa, and at room temperature, i.e. about 25° C., or a temperature which is established on combination of the components at room temperature without additional heating or cooling. All viscosity figures given in the examples are based on a temperature of 25° C.

The particle size measurement of the polyorganosiloxane emulsions according to the invention is carried out using a Malvern Mastersizer 2000 (Malvern Instruments GmbH, Herrenberg, Germany; measurement principle: Fraunhofer diffraction according to ISO 13320). A parameter assumed for the measurement is a spherical model with a refractive index of the continuous phase of 1.33, a refractive index of the disperse phase of 1.39 and an absorption of 0.01. The measured value reported is the D(50), the median of the volume distribution.

To determine the oil viscosity, 20 g of polyorganosiloxane emulsion is admixed with 30 g of acetone, causing the emulsion to separate. The acetone-water phase is removed and the procedure is repeated once more. Subsequently, the polymer is washed three times with water and dried at 110° C. while stirring until no water droplets are visible any longer, and then aftertreated at 110° C. in a drying cabinet for another 8 h.

The viscosities of the polyorganosiloxane emulsions and of the removed oil for the determination of the oil viscosity are determined at 25° C. in accordance with DIN 53019 using an MCR 302 rheometer from Anton Paar GmbH Graz. The CP50-2 cone-plate system is used and a flow curve is measured with shear rates in the range from 0.1 to 100/s. In the examples, the viscosity is reported at 10/s. To determine the content of octamethylcyclotetrasiloxane (D₄), 0.5 g of the sample is mixed with 10 ml of ultrapure acetone, which contained 120 ppm of n-dodecane as internal standard. After shaking for a period of 16 hours, two phases are formed. 10 μl of the supernatant clear phase is injected into a gas chromatograph. A double determination is carried out. The content of D₄ in the emulsion is determined on the basis of a calibration curve recorded beforehand. The D₄ content can then be calculated therefrom.

For the determination of the instability index, the emulsions are centrifuged for 8 h at 4000 rpm (corresponding to approx. 2300×g), using a LUMiFuge® 110.2-69 (LUM GmbH Berlin) and cuvettes with path lengths of 2 mm. The stability is characterized by the instability index output by the device software. If this is 0, the emulsion is stable; if it is 1, the emulsion is completely separated (cf. in this respect Dispersion Letters Technical, T4 (2013) 1-4, Update 2014). This means that the smaller this value, the more stable the emulsion.

The combing force of wet hair is determined using hair tresses from damaged, Caucasian hair from Kerling International Haarfabrik GmbH (hair tress degree of damage B, double drawn) with a weight of 2 g and a length of 20 cm. The combing force is measured by the double comb method in accordance with Y. K. Kamath and Hans-Dietrich Weigmann, J. Soc. Cosmet. Chem., 37, 111-124, 1986, using an Instron 3343 tensile tester. First, the wet combing force is determined along the measurement section on untreated hair tresses. The hair tresses are then treated with a cosmetic composition according to the invention and the force absorption during the combing procedure is determined. The measured value reported is the reduction in combing force along the measurement section (work) that arises between the treated and untreated hair tress. The average is formed from five hair tresses. The reduction in combing force is reported in percent.

### Raw Materials Used

Polyorganosiloxane A1: A trimethylsiloxy group-terminated polydimethylsiloxane having a viscosity of 60 000 mPa·s

Polyorganosiloxane A2 Mixture of trimethylsiloxy group-terminated polydimethylsiloxanes having a viscosity of 300 000 mPa·s and 1000 mPa·s; the mixture has a viscosity of 60 000 mPa·s.

Polyorganosiloxane A3: A hydroxyl group-terminated polydimethylsiloxane having a viscosity of 80 000 mPa·s

Polyorganosiloxane A4: A trimethylsiloxy group-terminated polydimethylsiloxane having a viscosity of 20 000 mPa·s

Polyorganosiloxane A5: A trimethylsiloxy group-terminated polydimethylsiloxane having a viscosity of 330 000 mPa·s

Alkyl polyglycoside B1: A 63% aqueous solution of a caprylyl/decyl glycoside, having a surface tension (1 g/l) of 29 mN/m and a pour point of less than 0° C., available under the name DISPONIL® APG 215 from BASF SE

Alkyl polyglycoside B2: A 53% aqueous solution of an alkyl polyglycoside C10-16 having a surface tension (1 g/l) of 28 mN/m and a pour point of 5° C., available under the name Glucopon® 600 CSUP from BASF SE

Alkyl polyglycoside B3: A 50% aqueous solution of a decyl octyl polyglycoside having a surface tension (1 g/l) of 29 mN/m and a pour point of 0° C., available under the name TRITON® CG-50 from Stockmeier Chemie GmbH Co KG Bielefeld

Further emulsifier D1: Sorbitan monolaurate available under the name SPAN® 20 from Croda GmbH Nettetal

Further emulsifier D2: Octyldecyl phosphate (acid value 330 mg KOH/g) available under the name CRODAFOS® 810 A from Croda GmbH Nettetal

Thickener E: Xanthan gum available under the name Xanthan FN from Jungbunzlauer Ladenburg GmbH Ladenburg

Further additives (preservatives) F1: K sorbate, benzoic acid and Na benzoate available from CSC JÄKLECHEMIE Gmbh & Co. KG Nuremberg

Further additives (preservatives) F2: Phenoxyethanol available from Thor GmbH Speyer

### Preparation of the Polyorganosiloxane Emulsions

The emulsions are prepared in an IKA® magic PLANT laboratory mixer from IKA® Werke Gmbh & Co. KG Staufen with an anchor stirrer with scrapers. The batch size in each case is 1500 g at 25° C. The formulations of Examples 1 to 9 according to the invention and of Comparative Examples C₁ to C₃ can be found in Table 1 below.

82.5 g of deionized water (C) is initially charged and components (B) and optionally (D) and optionally (E) are added. They are mixed for 10 min at a stirrer speed of 75/min (corresponding to a peripheral speed of 0.63 m/s). Component (A) is metered in over the course of 60 min and mixing is performed for a further 60 min at 75/min. The remaining water is added over the course of 60 min and stirred in at 75/min. In all experiments, 1.5 g of K sorbate, 1.95 g of benzoic acid 4.5 g of Na benzoate as component (F) (preservatives F1) are added at the end and stirred in for 30 min.

TABLE 1
Formulations¹⁾ of Examples 1 to 9 and Comparative Examples
C1 to C3 for components (A), (B), (D) and (E)

| | Component | | | | |
|---|---|---|---|---|---|
| | | | Parts of (B) per | | |
| Example | (A) | (B)²⁾ | 100 parts of (A) | (D) | (E) |
| 1 | 75% A1 | 2.90% B1 | 3.87 | — | — |
| 2 | 60% A1 | 2.52% B1 | 4.20 | — | 0.35% |
| 3 | 60% A2 | 2.52% B1 | 4.20 | 0.12% | 0.25% |
| | | | | D1³⁾ | |
| 4 | 60% A1 | 2.65% B2 | 4.42 | — | 0.20% |
| 5 | 60% A1 | 0.94% B1 | 1.57 | — | 0.35% |
| 6 | 60% A3 | 1.89% B1 | 3.15 | — | 0.35% |
| 7 | 60% A4 | 1.89% B1 | 3.15 | — | 0.35% |
| 8 | 60% A5 | 1.89% B1 | 3.15 | — | 0.35% |
| 9 | 60% A1 | 1.89% B3 | 3.15 | — | 0.35% |
| C1 | 60% A1 | 9.45% B1 | 15.75 | — | — |
| C2 | 60% A1 | 15.75% B1 | 26.25 | — | — |
| C3 | 60% A1 | 15.75% B1 | 26.25 | — | 0.10% |

¹⁾Use amounts of preservatives F1 as described above; water (C) must be added to 100% in each case.
²⁾Amount based on 100% active content
³⁾corresponds to 0.2 parts of (D) per 100 parts of (A)

In Table 2 below, Examples 1 to 9 according to the invention show only very slight signs of the beginning of a separation (instability index of less than 0.1) for the measurement of the instability index after 8 h at 2300×g, whereas non-inventive Examples C1 to C3, having higher emulsifier proportions, exhibit a similar viscosity but show significant signs of a separation with instability indices of greater than 0.3, or even greater than 0.5.

TABLE 2
Testing of the emulsion properties

| | | Viscosity in | |
|---|---|---|---|
| Example | D(50) in μm | mPa · s | Instability index |
| 1 | 2.75 | 1,344 | 0.075 |
| 2 | 6.48 | 2,310 | 0.026 |
| 3 | 4.62 | 1,510 | 0.080 |
| 4 | 4.08 | 2,070 | 0.052 |
| 5 | 8.00 | 2,350 | 0.016 |
| 6 | 5.91 | 2,610 | 0.020 |
| 7 | 3.36 | 2,310 | 0.013 |
| 8 | 9.89 | 2,740 | 0.029 |
| 9 | 6.53 | 2,360 | 0.021 |
| C1 | 1.09 | 1,580 | 0.305 |
| C2 | 1.20 | 1,740 | 0.540 |
| C3 | 7.34 | 2,810 | 0.518 |

### Example 10: Emulsion Polymerization with an Inventive Polyorganosiloxane Emulsion

82.5 g of deionized water (C) is initially charged and 60 g of B1 (corresponding to 37.8 g active content) and 5.25 g of E are added and mixing is performed for 10 min at a stirrer speed of 75/min (corresponding to a peripheral speed of 0.63 m/s). 895.65 g of component A3 is metered in over the course of 60 min and mixing is performed for a further 60 min at 75/min. Use is thus made of 4.22 parts of (B) per 100 parts of (A). The remaining water is added over the course of 60 min and stirred in at 75/min. 1.79 g of component D2 is then added and stirred in for 30 min. The emulsion is stored for 48 h at 4° C. and neutralized with 31.5 g of triethanolamine. 13.5 g of phenoxyethanol is then added as component (F) (preservative F2).

### Comparative Example C₄: Emulsion Polymerization with a Non-Inventive Polyorganosiloxane Emulsion

82.5 g of deionized water (C) is initially charged and 375 g of B1 (corresponding to 236.2 g active content) is added and mixing is performed for 10 min at a stirrer speed of 75/min (corresponding to a peripheral speed of 0.63 m/s). 895.65 g of component A3 is metered in over the course of 60 min and mixing is performed for a further 60 min at 75/min. Use is thus made of 26.37 parts of (B) per 100 parts of (A). The remaining water is added over the course of 60 min and stirred in at 75/min. 35 g of component D2 is then added and stirred in for 30 min. The emulsion is stored for 48 h at 4° C. and neutralized with 31.5 g of triethanolamine. 13.5 g of phenoxyethanol is then added as component (F) (preservative F2).

TABLE 3
Testing of the emulsion properties of
Example 10 and Comparative Example C4

| | | | | | D₄ in % by |
|---|---|---|---|---|---|
| | | Viscosity | Viscosity | | weight |
| | | of the | of the | | based on the |
| | D(50) | emulsion | oil phase | Instability | total weight of |
| Example | in μm | in mPa · s | in mPa · s | index | the emulsion |
| 10 | 5.53 | 2,680 | 2,165,000 | 0.019 | 0.018 |
| C4 | 1.76 | 2,880 | 110,372 | 0.158 | not determined |

As Table 3 shows, the inventive polyorganosiloxane emulsion of Example 10 enables the formation of a very high-viscosity oil without impairing the stability of the emulsion and without forming significant amounts of octamethylcyclotetrasiloxane (D₄).

Only a slight increase in the oil viscosity occurs in the non-inventive polyorganosiloxane emulsion from Comparative Example C₄. Therefore, there is also no test for the D₄ content. The instability index is significantly worse despite the much smaller particle size and slightly higher viscosity.

### Example 11: Application Example as Hair Care Product

A shampoo is formulated from the components shown in Table 4:

TABLE 4
Shampoo formulation components

| | Amount in |
|---|---|
| | % by |
| Constituents (INCI* name) | weight |
| Citric Acid¹⁾ | 0.05 |
| Cocamidopropyl Betaine²⁾ | 5.00 |
| Sodium Laureth Sulfate³⁾ | 29.90 |
| Guar Hydroxypropyltrimonium Chloride⁴⁾ | 0.20 |
| Sodium Lauryl Sulfate⁵⁾ | 6.06 |
| Aqua (DI water) | 33.65 |
| Carbomer⁶⁾ | 0.60 |
| Lactic Acid⁷⁾ | 0.06 |
| Aqua (DI water) | 20.00 |
| Phenoxyethanol, Ethylhexylglycerin⁸⁾ | 0.95 |
| C12-13 Alkyl Lactate⁹⁾ | 0.30 |
| Polyorganosiloxane emulsion from Example 2 or C2 | 2.17 |
| Sodium Hydroxide¹⁰⁾ | 0.40 |
| Sodium Chloride¹¹⁾ | 0.66 |

*International Nomenclature of Cosmetic Ingredients
The raw materials specified in Table 4 are available under the following trade names:
¹⁾Citric Acid, Sigma
²⁾Genagen ® CAB 818 30%, Clariant
³⁾Genapol ® LRO 26, 5%, Clariant
⁴⁾N-Hance ® BF 13, Ashland
⁵⁾Texapon ® K 12 G, BASF
⁶⁾Carbopol ® 980, Lubrizol
⁷⁾L-(+)Lactic acid, 90%, Bernd Kraft GmbH
⁸⁾Phenoxyethanol, Ethylhexylglycerin: Euxyl ® PE 9010, Schülke & Mayr
⁹⁾Ceraphyl ™ 41 Ester, Ashland
¹⁰⁾Sodium hydroxide, Sigma-Aldrich
¹¹⁾Sodium chloride ultrapure, Bernd Kraft GmbH

In comparison with a shampoo without silicone emulsion, the wet combing force on Caucasian hair is reduced by 30%.

If the non-inventive emulsion of Example C2 is used instead of the inventive emulsion of Example 2, the wet combing force is reduced only by 10%.

1-17. (canceled)

18. A process for preparing storage-stable, aqueous polyorganosiloxane emulsions comprising
(A) at least one polyorganosiloxane of the general formula 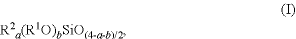
where
R¹ may be identical or different and is a hydrogen atom or a monovalent, substituted or unsubstituted hydrocarbon radical,
R² may be identical or different and is a monovalent, substituted or unsubstituted hydrocarbon radical having 1 to 30 carbon atoms or a hydrogen atom,
a is 0, 1, 2 or 3,
b is 0, 1, 2 or 3,
with the proviso that the sum total of a+b is ≤3,
(B) at least one alkyl polyglycoside of the general formula 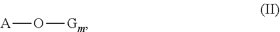
where
A is a linear or branched, saturated or unsaturated alkyl radical having 8 to 24 carbon atoms,
G is a glycoside radical and
m is the degree of glycosidation and is an integer of on average 1 to 5,
(C) water,
optionally
(D) further emulsifiers that are different from the alkyl polyglycosides (B),
optionally
(E) thickeners
and optionally
(F) further additives that are usually present in polyorganosiloxane emulsions,
characterized in that
(v) a total of 1.5 to 4.5 parts of alkyl polyglycoside (B) are present per 100 parts of polyorganosiloxane (A),
(vi) a total of 0 to 3 parts of further emulsifiers (D) are present per 100 parts of polyorganosiloxane (A),
(vii) the sum total of the parts of alkyl polyglycoside (B) and further emulsifiers (D) is ≤4.5 per 100 parts of polyorganosiloxane (A)
and
(viii) the process comprises the following steps
(5) preparation of an emulsifier-water mixture comprising
at least one alkyl polyglycoside (B) and water (C) and optionally further emulsifiers (D), optionally thickeners (E) and optionally further additives (F) preparation of a preliminary emulsion by addition of
(6)
at least one polyorganosiloxane (A) to the emulsifier-water mixture, with the proviso that the preliminary emulsion is prepared under the action of low shear forces using stirring devices with maximum peripheral speeds of 4.5 m/s,
optionally
(7) dilution of the preliminary emulsion by addition of
further water (C)
and optionally
(8) addition of further additives (F),
where the process yields polyorganosiloxane emulsions having a particle size D(50) of 1 μm to 50 μm, based on the D(50) value of the volume distribution measured in accordance with the principle of Fraunhofer diffraction (according to ISO 13320),
and in that polydimethylsiloxanes having a viscosity of 10 000 mPa·s to 3 000 000 mPa's (determined at 25° C. in accordance with DIN 53019) are used as polyorganosiloxanes (A).

19. The process as claimed in claim 18, wherein alkyl polyglycosides (B) having a saturated alkyl radical having an average of 8 to 14 carbon atoms and an average degree of glycosidation m of 1.1 to 3.0 are used.

20. The process as claimed in claim 18, wherein further emulsifiers (D) are not present.

21. The process as claimed in claim 18, wherein 0.1 to 1 part(s) of xanthan gum per 100 parts of polyorganosiloxane (A) are used as thickener (E).

22. The process as claimed in claim 18, wherein the instability index of the polyorganosiloxane emulsions, after centrifugation at 2300×g for 8 h, is <0.1.

23. The process as claimed in claim 18, wherein the polyorganosiloxane emulsions have a particle size D(50) of 3 μm to 20 μm, based on the D(50) value of the volume distribution measured in accordance with the principle of Fraunhofer diffraction (according to ISO 13320).

24. The process as claimed in claim 18, wherein the polyorganosiloxane emulsions have a content of octamethylcyclotetrasiloxane (D₄) of less than 0.1% by weight, based on the total weight of the polyorganosiloxane emulsion.

25. Storage-stable, aqueous polyorganosiloxane emulsions comprising
(A) at least one polyorganosiloxane of the general formula 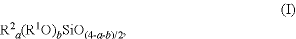
where
R¹ may be identical or different and is a hydrogen atom or a monovalent, substituted or unsubstituted hydrocarbon radical,
R² may be identical or different and is a monovalent, substituted or unsubstituted hydrocarbon radical having 1 to 30 carbon atoms or a hydrogen atom,
a is 0, 1, 2 or 3,
b is 0, 1, 2 or 3,
with the proviso that the sum total of a+b is ≤3,
(B) at least one alkyl polyglycoside of the general formula 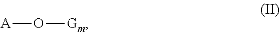
where
A is a linear or branched, saturated or unsaturated alkyl radical having 8 to 24 carbon atoms,
G is a glycoside radical and
m is the degree of glycosidation and is an integer of on average 1 to 5,
(C) water,
optionally
(D) further emulsifiers that are different from the alkyl polyglycosides (B),
optionally
(E) thickeners
and optionally
(F) further additives that are usually present in polyorganosiloxane emulsions,
characterized in that
(i) a total of 1.5 to 4.5 parts of alkyl polyglycoside (B) are present per 100 parts of polyorganosiloxane (A),
(ii) a total of 0 to 3 parts of further emulsifiers (D) are present per 100 parts of polyorganosiloxane (A),
(iii) the sum total of the parts of alkyl polyglycoside (B) and further emulsifiers (D) is ≤4.5 per 100 parts of polyorganosiloxane (A)
and
(iv) the emulsions are produced by a process comprising the steps
(1) preparation of an emulsifier-water mixture comprising at least one alkyl polyglycoside (B) and water (C) and optionally further emulsifiers (D), optionally thickeners (E) and optionally further additives (F),
(2) preparation of a preliminary emulsion by addition of at least one polyorganosiloxane (A) to the emulsifier-water mixture, with the proviso that the preliminary emulsion is prepared under the action of low shear forces using stirring devices with maximum peripheral speeds of 4.5 m/s,
optionally
(3) dilution of the preliminary emulsion by addition of further water (C)
and optionally
(4) addition of further additives (F),
with the proviso that the polyorganosiloxane emulsions have a particle size D(50) of 1 μm to 50 μm, based on the D(50) value of the volume distribution measured in accordance with the principle of Fraunhofer diffraction (according to ISO 13320),
and in that polydimethylsiloxanes having a viscosity of 10 000 mPa's to 3 000 000 mPa's (determined at 25° C. in accordance with DIN 53019) are used as polyorganosiloxanes (A).

26. The polyorganosiloxane emulsions as claimed in claim 25, wherein alkyl polyglycosides (B) having a saturated alkyl radical having an average of 8 to 14 carbon atoms and an average degree of glycosidation m of 1.1 to 3.0 are used.

27. The polyorganosiloxane emulsions as claimed in claim 25, wherein further emulsifiers (D) are not present.

28. The polyorganosiloxane emulsions as claimed in claim 25, wherein 0.1 to 1 part(s) of xanthan gum per 100 parts of polyorganosiloxane (A) are used as thickener (E).

29. The polyorganosiloxane emulsions as claimed in claim 25, wherein the instability index of the polyorganosiloxane emulsions, after centrifugation at 2300×g for 8 h, is <0.1.

30. The polyorganosiloxane emulsions as claimed in claim 25, wherein the polyorganosiloxane emulsions have a particle size D(50) of 3 μm to 20 μm, based on the D(50) value of the volume distribution measured in accordance with the principle of Fraunhofer diffraction (according to ISO 13320).

31. The polyorganosiloxane emulsions as claimed in claim 25, wherein the polyorganosiloxane emulsions have a content of octamethylcyclotetrasiloxane (D₄) of less than 0.1% by weight, based on the total weight of the polyorganosiloxane emulsion.

32. A cosmetic preparation comprising a polyorganosiloxane emulsion of claim 25.
